(19) United States
(12) Patent Application Publication
(10) Pub. No.: US 2007/0042999 A1
(43) Pub. Date: Feb. 22, 2007

(54) Phosphate derivatives of pharmaceutical products

(76) Inventors: Simon Michael West, Williamstown (AU); David Kannar, Belgrave South (AU)

Correspondence Address: REED SMITH LLP, P.O. BOX 488, PITTSBURGH, PA, 15230-0488, US

(21) Appl. No.: 10/551,201

(22) PCT Filed: Apr. 14, 2004
(86) PCT No.: PCT/AU04/00492

§ 371 Date: Jul. 24, 2006

(30) Foreign Application Priority Data
Apr. 15, 2003 (AU) 2003901813

(51) Int. Cl. A61K 31/675; A61K 31/665; C07J 51/00; C07F 9/06; C07F 9/576; C07F 9/6547

(52) U.S. Cl. 514/81; 514/100; 514/114; 514/125; 546/22; 552/506; 549/218; 549/220

(57) ABSTRACT

According to the invention, there is provided a complex of a pharmaceutical compound selected from the group consisting of opioids, hormones, anaethetics and chemotherapeutic agents comprising the reaction product of: (a) one or more phosphate derivatives of one or more opioids, steroid hormones, thyroid hormones, anaesthetics or chemotherapeutic agents having a phenolic, primary alcohol, secondary alcohol or tertiary hydroxyl group; and (b) a complexing agent selected from the group comprising amphoteric surfactants, cationic surfactants, amino acids having nitrogen functional groups and proteins rich in these amino acids.

### FIELD OF THE INVENTION

The invention relates to phosphate derivatives of opioid analgesics, chemotherapeutics, anaesthetics and hormones.

### BACKGROUND OF THE INVENTION

In this specification, where a document, act or item of knowledge is referred to or discussed, this reference or discussion is not an admission that the document, act or item of knowledge or any combination thereof was at the priority date part of common general knowledge; or known to be relevant to an attempt to solve any problem with which this specification is concerned.

Whilst the present invention will be described with reference to specific compounds such as opium, morphine, testosterone, thyroxine or alfaxalone, it should be understood that the present invention is not so limited but applies more generally to opioid analgesics, chemotherapeutics, anaesthetics and hormones having a phenolic primary alcohol, secondary alcohol or tertiary alcohol group.

Opioid Analgesics

Opium is obtained from the opium poppy, Papaver somniferum, by incision of the seed pod after petals of the flower have dropped. This raw material contains approximately 20 alkaloids including morphine, codeine, thebaine and papaverine. These compounds are commonly called opioids. The term ‘opioid’ refers to any natural or synthetic drug that has morphine-like pharmacological actions and is a term used interchangeably with ‘narcotic analgesic’.

Opioids produce central nervous system analgesia by acting on regions of the brain containing peptides that are also known to have opioid-like properties. These nascent compounds are known as “endogenous opioid peptides” and were formerly called “endorphins”. Opioid agonists bind to specific opioid receptors in the brain and spinal cord involved in the modulation and transmission of pain. This action has been clinically exploited by delivery of the agonist directly into the spinal cord, which not only provides a regional analgesic effect but also minimizes unwanted side effects such as respiratory depression, nausea, vomiting and sedation that may occur with systemic delivery. Opioids have also been reported to act locally most likely through binding to peripheral opioid receptors of inflamed tissue, but the actual mechanism is unknown.

Opioid Derivatives

Morphine has the following structure:

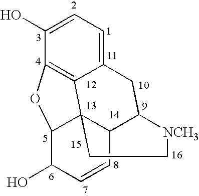

The chemical structure of the opioid compound determines the action of the drug. Importantly, substitutions at the C₃ and C₆ hydroxyl groups of morphine significantly alter its pharmacokinetics (see table below). Methylation of the phenolic hydroxyl at C₃ reduces first pass metabolism by glucuronide conjugation. Drugs methylated in this manner such as codeine and oxycodone also have a higher oral than parenteral potency because of protection of the hydroxyl group by the methyl group. Acetylation of both hydroxyl groups produces heroin and dramatically improves penetration across the blood brain barrier causing a euphoric but also produces highly addictive effects. Analgesic activity is reported to improve with conjugation of the hydroxyl groups in the following decreasing order: sulfate>glucuronide=acetate>phosphate>morphine.

Chemical radicals at
key positions (see above
structure for positions)

| Trivial name | C3 | C6 |
|---|---|---|
| Heroin | —OCOCH₃ | —OCOCH₃ |
| Hydromorphone | —OH | ═O |
| Oxymorphone | —OH | ═O |
| Levorphanol | —OH | —H |
| Codeine | —OCH₃ | —OH |
| Hydrocodone | —OCH₃ | ═O |
| Oxycodone | —OCH₃ | ═O |
| Nalorphine | —OH | —OH |
| Naloxone | —OH | ═O |

(Note that there may be other substituent changes which have not been mentioned)

Routes of Administration

Most opioids are well absorbed from subcutaneous tissue, intramuscular sites, and mucosal surfaces of the nose and mouth, although transdermal administration is not the preferred route of administration for most opioids.

Absorption of opioids through the gastrointestinal tract is also thought to be rapid, but highly variable if the opioid drug is subject to first pass metabolism. This variability is thought to be due to the wide variation in glucuronidase activity between individuals. Therefore, in some cases the oral dose required to elicit a therapeutic effect may be higher than the parenteral dose.

There is a need to increase absorption of opioids from various administration routes and to improve efficacy of opioid drugs.

Steroid Hormones

Whilst the following discussion relates to testosterone, it will be understood that the invention has applications to other steroid hormones where improved delivery is desired.

Although testosterone and other active steroid hormones can be isolated in pure form, their effect is still measured in biological assays. The specific biologically active form therefore has not been identified. Steroid phosphates have been considered as potential members of biological systems but have not been isolated from animal tissues or body fluids. In vitro biosynthesis of estrogen phosphates have however been reported in rat liver and are known to be substrates for alkaline and acid phosphates extracted from various animal tissues. This indicates that phosphorylated steroid hormones could be intermediate compounds and a natural storage form.

According to pharmaceutical literature, orally delivered charged compounds such as steroid phosphates will not be bioavailable and of little value because;
(a) highly ionized species do not readily undergo passive diffusion across cellular membranes and
(b) phosphates, particularly those of primary alcohols and phenols, are known to be substrates for many phosphorylases present in the body which readily clip the phosphate group from the drug resulting in a short duration of action.

In humans, the most important androgen is testosterone, as it is responsible for the many changes that occur in the normal male at puberty. When administered orally, testosterone is rapidly absorbed but largely converted to inactive metabolites, with less than one sixth of the administered dose being available in the active form. To improve its delivery derivatised testosterone analogues have been produced.

Esterified forms including propionate, enanthate, undecanoate or cypionate, have prolonged absorption time and greater activity. Mixed testosterone esters in a vegetable oil vehicle are used for intramuscular injection. This formulation acts as a depot preparation. Once released from the depot the testosterone ester is rapidly hydrolysed at the site of injection. The pharmacokinetics of these formulations are dependant upon the ester side-chain length and hydrophobicity, which determine the kinetics of release from the oil vehicle.

Unmodified testosterone is also used in a number of formulations. Fused pellets of crystalline testosterone provide stable physiological blood levels but the implantation procedure and its complications limit its utility. Transdermal patches can also maintain physiological levels but require the addition of absorption enhancers that can potentially irritate the skin. Scrotal patches take advantage of the thin and highly vascular skin of the scrotum but still require a large surface area for absorption. Dermal administration is therefore less than optimal.

Testosterone undecanoate is administered in an oleic acid suspension orally. This formulation enhances chylomicron absorption but has low and erratic bioavailability. Sublingual testosterone raises blood levels for a short period of time and is therefore required to be administered many times a day, making it unsuitable for long term replacement. Micronised testosterone has low oral bioavailability and high doses are thus required to maintain physiological levels. These high doses cause significant hepatic enzyme induction and are therefore not favoured. Oral, dermal and delivery of testosterone by other routes of administration are therefore currently less than optimal.

Thyroid Hormones

Thyroid hormones set the body's metabolic rate and are essential for growth and development. They have wide ranging effects on all body systems and are vital for development of nervous, skeletal and reproductive tissues. Its effects however depend upon protein synthesis, potentiation of secretion and action of growth hormone. Thyroid hormones bind to proteins and enter the cell by diffusion and/or possibly active transport processes.

The normal thyroid gland produces sufficient amounts of the thyroid hormone to maintain normal growth and development, normal body temperature and energy levels. When under produced, for whatever reason, the effects are known as hypothyroidism. Hypothyroidism in developing children can lead to mental deficiency and the syndrome of hypothyroidism known as cretinism. Treatment of hypothyroidism is by hormone replacement. The thyroid hormone currently known is L-Thyroxine, phosphate (6CI) (CAS 108851-05-4).

There are 4 different forms of thyroid hormone available for replacement—thyroxine (T₄), triiothyronine (T₃), thyroglobulin and desiccated thyroid. Thyroxine and triiothyronine contain 65 and 59% iodine as an essential part of the molecule. Thyroxine is the most commonly prescribed method of treatment. Triiothyronine may have a place in limited and rare circumstances but there is no longer a place for thyroglobulin and desiccated thyroid in clinical management of hypothyroidism.

Thyroxine is rapidly absorbed from the gut, in the duodenum and ileum. Absorption, however, is variable with bioavailability ranging from 50-80% and modified by intraluminal factors such as food, drugs (aluminium containing antacids, sucralfate, and iron) and intestinal flora. Differing generic formulations of thyroxine are not generally considered interchangeable due to the variability of absorption.

Thyroid hormone does not readily cross the placenta nor is it excreted to any great degree in breast milk. This means that the mother cannot compensate adequately for a lack of foetal hormone production. Varying formulations of thyroid hormone have been studied to attempt to find a form that will cross the placenta but with limited success.

Paclitaxel

Paclitaxel is an alkaloid ester derived from the Western and European yew trees (Taxus brevifolia & baccata) and highly toxic compound with demonstrated clinical antitumor efficacy. Paclitaxel has an unusual mechanism involving stabilization of core structural proteins necessary for assembly and disassembly of mitotic spindles called tubulin polymerization. Stabilisation of tubulin polymerization effectively inhibits uncontrolled tumor stem cellular division leading to metastasis.

Paclitaxel is very lipidic and difficult to formulate, requiring use of lipid co-solvents that are thought to cause their own side effects. This results in a major clinical problem when using paclitaxel as an intravenous anticancer agent. Derivatives of paclitaxel possessing a phosphate moiety at positions C-2′ and C-7 have been reported, but neither compound possesses in vitro tubulin activity nor in vivo antitumor efficacy. In contrast C-2′ and C-7 phosphonoxyphenylpropionate paclitaxel derivatives both generated paclitaxel after treatment with alkaline phosphatase but only the C-7 analogue had comparable antitumor efficacy to paclitaxel in an M109 murine lung carcinoma model.

Important disadvantages of paclitaxel arise from its lipid solubility. The compound therefore need to be delivered in other more soluble lipidic carriers that improve their dissolution. Paclitaxel is dissolved in a medium chain length triglyceride (Cremophor), oil in water emulsions (Intralipid), polyoxyl 35 castor oil (hydrogenated castor oil) or other lipidic emulsion systems.

Hypersensitivity reactions have been reported using these delivery systems, including hypotension, flushing and bronchospasm, but are largely thought to be due to the lipid vehicle Cremaphor. Although side effects using intralipid emulsions are reported to be lower, an improved delivery strategy needs to be developed.

While the phosphonoxyphenylpropionate derivates may be more water soluble than the parent compound they are still likely to require administration with lipidic co-solvents and is of limited benefit. A complex that quickly dissociates and reverts to the parent compound yet is water soluble would be preferred.

Anesthetic—Alfaxalone

An ideal anaesthetic drug would induce anesthesia smoothly and quickly, then permit rapid recovery upon cessation. The drug would also be safe to use and free of side effects, but as no single agent possesses all these attributes, combinations of drugs are often used in modern practice.

The anesthetic considered in this application is a major veterinary product alfaxalone. Clinical utility of this intravenous administered compound is marred by poor solubility. This complicates formulation of the drug. A phosphate derivative is known for alfaxalone (CAS 2428-88-8). Although phosphate pro-drugs of these compounds are water soluble, rapid conversion of alfaxalone phosphate to the parent drug following intravenous administration may not be achieved in vivo. These include hypotension, flushing and bronchospasm, but are largely thought to be due to the lipid vehicle Cremaphor. Although side effects using intralipid emulsions are reported to be lower, an improved delivery strategy needs to be developed.

### SUMMARY OF THE INVENTION

According to a first aspect of the invention, there is provided a complex of a pharmaceutical compound selected from the group consisting of opioids, hormones, anaethetics and chemotherapeutics agents, the derivative comprising the reaction product of:
(c) one or more phosphate derivatives of one or more opioids, steroid hormones, thyroid hormones, anaesthetics or chemotherapeutic agents having a phenolic, primary alcohol, secondary alcohol or tertiary hydroxyl group; and
(d) a complexing agent selected from the group comprising amphoteric surfactants, cationic surfactants, amino acids having nitrogen functional groups and proteins rich in these amino acids.

Preferably, where irritation may be caused upon administration of the complex, it is administered in a formulation comprising an effective amount of the reaction product of:
(a) one or more phosphate derivatives of tocopherol; and
(b) a complexing agent selected from the group comprising amphoteric surfactants, cationic surfactants, amino acids having nitrogen functional groups and proteins rich in these amino acids.

According to a second aspect of the invention, there is provided a phosphatidyl derivative of a pharmaceutical compound selected from the group consisting of opioid, steroid hormones, thyroid hormones, anaesthetics or chemotherapeutic agents having a phenolic, primary alcohol, secondary alcohol or tertiary hydroxyl group.

According to a third aspect of the invention, there is provided a method for preparation of a phosphate derivative of a pharmaceutical compound selected from the group consisting of opioids, steroid hormones, thyroid hormones, anaesthetics or chemotherapeutic agents having a phenolic, primary alcohol, secondary alcohol or tertiary hydroxyl group comprising the step of reacting the pharmaceutical compound with P₄O₁₀ in the presence of a sodium salt of a fatty acid.

Preferably, the method further comprising the step of reacting the product from the P₄O₁₀ reaction with a di or mono acyl glyceride to form a phosphatide.

According to a further aspect of the invention there is provided use of a phosphate derivative of a pharmaceutical compound selected from the group consisting of opioids, steroid hormones, thyroid hormones, anaesthetics or chemotherapeutic agents having a phenolic, primary alcohol, secondary alcohol or tertiary hydroxyl group to make medicaments for use in treating humans or animals.

Where used herein the term “phosphate derivatives” refers to compounds covalently bound by means of an oxygen to the phosphorus atom of a phosphate group. The phosphate derivative may exist in the form of a free phosphate acid, a salt thereof, a di-phosphate ester thereby including two one or more opioids, steroid hormones, thyroid hormones, anaesthetics or chemotherapeutic agents having a phenolic, primary alcohol, secondary alcohol or tertiary hydroxyl group molecules, a mixed ester including two different compounds selected from opioids, steroid hormones, thyroid hormones, anaesthetics or chemotherapeutic agents having a phenolic, primary alcohol, secondary alcohol or tertiary hydroxyl group, and a phosphatidyl compound wherein the free phosphate oxygen forms a bond with an alkyl or substituted alkyl group.

Suitable complexing agents for use in the present invention may be selected from surfactants chosen from the classes including alkyl amino/amido betaines, sultaines, phosphobetaines, phosphitaines, imidazolimum and straight chain mono and dicarboxy ampholytes, quaternary ammonium salts, and cationic alkoxylated mono and di-fatty amines; and amino acids having nitrogen functional groups and proteins rich in these amino acids. Preferred complexing are agents N-lauryl imino di-propionate and arginine.

Suitable amino acids having nitrogen functional groups for use in the present invention include glycine, arginine, lysine and histidine. Proteins rich in these amino acids may also be used as complexing agents, for example, casein. These complexing agents are used when the composition needs to be orally ingestible.

The amphoteric surfactants may be ampholytic surfactants, that is, they exhibit a pronounced isoelectric point within a specific pH range; or zwitterionic surfactants, that is, they are cationic over the entire pH range and do not usually exhibit a pronounced isoelectric point. Examples of these amphoteric surfactants are tertiary substituted amines, such as those according to the following formula:
NR¹R²R³
wherein R¹ is chosen from the group comprising straight or branched chain mixed alkyl radicals from C6 to C22 and carbonyl derivatives thereof.

R² and R³ are independently chosen from the group comprising H, CH₂COOX, CH₂CHOHCH₂SO₃X, CH₂CHOHCH₂OPO₃X, CH₂CH₂COOX, CH₂COOX, CH₂CH₂CHOHCH₂SO₃X or CH₂CH₂CHOHCH₂OPO₃X and X is H, Na, K or alkanolamine provided that R² and R³ are not both H.

In addition, when R¹ is RCO then R² may be CH₃ and R³ may be (CH₂CH₂)N(C₂H₄OH)—H₂CHOPO₃ or R² and R³ together may be N(CH₂)₂N(C₂H₄OH)CH₂COO—.

Commercial examples are DERIPHAT sold by Henkel/Cognis, DEHYTON sold by Henkel/Cognis, TEGOBETAINE sold by Goldschmidt and MIRANOL sold by Rhone Poulenc.

Cationic surfactants, such as quaternary ammonium compounds, will also form complexes with phosphorylated derivatives of drug hydroxy compounds such as tocopheryl phosphates. Examples of cationic surfactants include the following:
(a) RN⁺(CH₃)₃ Cl⁻
(b) [R₂N⁺CH₃]₂SO₄²⁻
(c) [(RCON(CH₃)CH₂CH₂CH₂N⁺(CH₃)₂C₂H₄OH]₂ SO₄²⁻
(d) Ethomeens: RN[(CH₂CH₂O)ₓCH₂OH][(CH₂CH₂O)_{y} CH₂OH] wherein x and y are integers from 1 to 50.
   wherein R is C8 to C22 straight or branched chain alkyl groups or mixed alkyl groups.

Silicone surfactants including hydrophilic and hydrophobic functionality may also be used, for example, dimethicone PG betaine, amodimethicone or trimethylsilylamodimethicone. For example, ABILE 9950 from Goldschmidt Chemical Co. The hydrophobe can be a C6 to C₂₂ straight—or branched alkyl or mixed alkyl including fluoroalkyl, fluorosilicone and or mixtures thereof. The hydrophilic portion can be an alkali metal, alkaline earth or alkanolamine salts of carboxy alkyl groups or sulfoxy alkyl groups, that is sultaines, phosphitaines or phosphobetaines or mixtures thereof.

Typically, the reaction product of the present invention is made by (1) direct neutralization of the free phosphoric acid ester of opioid, steroid hormones, thyroid hormones, anaesthetics or chemotherapeutic agents having a phenolic, primary alcohol, secondary alcohol or tertiary hydroxyl group with the complexing agents or (2) in-situ blending of mixed sodium salts of the phosphate derivatives of opioid, steroid hormones, thyroid hormones, anaesthetics or chemotherapeutic agents having a phenolic, primary alcohol, secondary alcohol or tertiary hydroxyl group with the complexing agents.

Examples of compounds which may be used in the invention include morphine (CAS 57-27-2), hydromorphone, oxymorphone, levorphanol, codeine, oxycodone, nalbuphine, buprenorphine, butorphanol, pentazocine, nalorphine (CAS 62-67-9), naloxone, naltrexone, levallorphan, levothyroxine (CAS 51-48-9), paclitaxel (CAS 33069-62-4), alfaxalone (CAS 23930-19-0), estradiol (CAS 50-28-2), estrone (CAS 53-16-7), estriol (CAS 50-27-1), ethinyl estradiol, progestins, methyltestosterone, testosterone (CAS 58-22-0), nandrolone (CAS 434-22-0) and danazol.

Opiod derivatives:
Morphine (CAS 57-27-2),
Heroin (diester),
Morphinan-3,6-diol, 7,8-didehydro-4,5-epoxy-17-methyl-(5.alpha.,6.alpha.)-, 6-(dihydrogen phosphate) (9CI) (common name morphine 6-phosphate) (CAS 51025-95-7),
Hydromorphone,
Morphinan-3,6-diol, 7,8-didehydro-4,5-epoxy-17-methyl-(5.alpha.,6.alpha.)-, 3-(dihydrogen phosphate) (9CI) (common name morphine 3-phosphate) (CAS 51065-90-8),
Oxymorphone,
Morphinan-6-one, 17-(cyclopropylmethyl)-4,5-epoxy-14-hydroxy-3-(phosphonooxy)-, disodium salt, (5.alpha.)- (9CI) (CAS 138618-00-5)
Levorphanol,
morphine hydrochloride,
Codeine,
morphine sulfate,
Oxycodone,
Morphinan-6-one, 17-(cyclopropylmethyl)-4,5-epoxy-14-hydroxy-3-(phosphonooxy)-, (5.alpha.)-(9CI) (CAS 156047-16-4)
Nalbuphine,
Morphinan-6-one, 4,5-epoxy-14-hydroxy-3-[(hydroxymethoxyphosphinyl)oxy]-17-(2-propenyl)-, (5.alpha.)- (9CI) (CAS 156047-24-4)
Pentazocine
Morphinan-6-one, 4,5-epoxy-14-hydroxy-3-(phosphonooxy)-17-(2-propenyl)-, (5.alpha.)-(9CI) (CAS 141843-94-9)
Butorphanol,
Morphinan-6-one, 4,5-epoxy-14-hydroxy-3-(phosphonooxy)-17-(2-propenyl)-, disodium salt, (5.alpha.)-(9CI) (CAS 138617-99-9)
buprenorphine,
Morphinan-6-one, 4,5-epoxy-14-hydroxy-3-[(hydroxymethoxyphosphinyl)oxy]-17-(2-propenyl)-, monosodium salt, (5.alpha.)-(9CI) (CAS 138617-97-7)
morphine glucuronide,

Steroid hormones:
Estra-1,3,5(10)-triene-3,17-diol (17β)-, 17-(dihydrogen phosphate), hydrate (9CI) (CAS 212623-59-1)
Estra-1,3,5(10)-triene-3,17-diol, 17-(dihydrogen phosphate), disodium salt, (17α)-(9CI) (CAS 182624-58-4)
Estra-1,3,5(10)-triene-3,17-diol (17β)-, 3-(dihydrogen phosphate), disodium salt (9CI) (CAS 136790-41-5)
Estra-1,3,5(10)-triene-3,17-diol (17β)-, 3-(dihydrogen phosphate), sodium salt (9CI) (CAS 66856-98-2)
Estra-1,3,5(10)-triene-3,17-diol (17β)-, 17-(dihydrogen phosphate), sodium salt (9CI) (CAS 66856-97-1)
Estra-1,3,5(10)-triene-3,17-diol (17β)-, 17-(dihydrogen phosphate), homopolymer (9CI) (CAS 34828-67-6)
Estradiol, mono(dihydrogen phosphate) (8CI) (CAS 27177-83-9)
Estra-1,3,5(10)-triene-3,17-diol (17β)-, 3-(dihydrogen phosphate) (9CI) (CAS 13425-82-6)
Estra-1,3,5(10)-triene-3,17-diol (17β)-, 17-(dihydrogen phosphate), disodium salt (9CI) (CAS 6345-23-9)
Estra-1,3,5(10)-triene-3,17-diol (17β)-, 17-(dihydrogen phosphate) (9CI) (CAS 4995-43-1)
Estra-1,3,5(10)-triene-3,17-diol, 3-(dihydrogen phosphate) (8CI, 9CI) (CAS 1098-52-8)
Androst-4-en-3-one, 17-(phosphonooxy)-, (17α)-(9CI)
Androst-5-en-17-one, 3β-hydroxy-, phosphate, dipotassium salt (7CI)
Androst-4-en-3-one, 17-(phosphonooxy)-, (17α)-(9CI)
Androst-4-en-3-one, 17-(phosphonooxy)-, disodium salt (9CI)
Androst-4-en-3-one, 17-(phosphonooxy)-, disodium salt, (17β)-(9CI)
Androst-4-en-3-one, 17-(phosphonooxy)-, (17β)-, compd. With N,N-diethylethanamine (9CI)
Androst-4-en-3-one, 17-(phosphonooxy)-, (17β)- (9CI)

Natural and synthetic estrogens, progestins, androgens, and antagonists and inhibitors:
danazol
Estr-4-en-3-one, 17-(phosphonooxy)-, disodium salt, (8α, 9β, 10α, 13α, 14β, 17α)-(9CI) (CAS 60700-27-8)
Estr-4-en-3-one, 17-(phosphonooxy)-, disodium salt, (17,β)-(9CI) (CAS 60672-81-3)
Estr-4-en-3-one, 17-(phosphonooxy)-, (8α, 9β, 10α, 13α, 14β, 17α)-(9CI) (CAS 29346-91-6)
Estr-4-en-3-one, 17-(phosphonooxy)-, (17β)-(9CI) or
Estr-4-en-3-one, 17β-hydroxy-, dihydrogen phosphate (7CI, 8CI) (CAS 1098-15-3) known as (+)-19-Nortestosterone 17-phosphate
Androst-4-en-3-one, 17-(phosphonooxy)-, disodium salt (9CI) (CAS 318481-34-4)
Androst-4-en-3-one, 17-(phosphonooxy)-, (17β)-, compd. With N,N-diethylethanamine (9CI) (194534-52-6)
Androst-4-en-3-one, 17-(phosphonooxy)-, (17α)-(9CI) (142546-96-1) Common Name 17-epi-Testosterone phosphate
Androst-4-en-3-one, 17-(phosphonooxy)-, disodium salt, (17β)-(9CI)***(CAS 67494-61-5) Common Name: Testosterone sodium phosphate
Androst-4-en-3-one, 17-(phosphonooxy)-, (17β)-(9CI) (CAS 1242-14-4) Common names: Testosterone phosphate (6CI) or Testosterone, dihydrogen phosphate (7CI, 8CI)

Paclitaxel forms:
Benzenepropanoic acid, β-(benzoylamino)-α-hydroxy-,6,12b-bis(acetyloxy)-12-(benzoyloxy)-2a,3,4,4a,5,6,9,10,11,12,12a, 12b-dodecahydro-11-hydroxy-4a,8,13,13-tetramethyl-5-oxo-4-(phosphonooxy)-7,11-methano-1H-cyclodeca[3,4]benz[1,2-b]oxet-9-yl ester, [2aR-[2aα,4β,4aβ,6β,9α(αR*,βS*),11α,12α,12aα,12bα]]-(9CI) (CAS 151765-63-8)
Benzenepropanoic acid, β-(benzoylamino)-α-(phosphonooxy)-,6,12b-bis(acetyloxy)-12-(benzoyloxy)-2a,3,4,4a,5,6,9,10,11,12,12a,12b-dodecahydro-4,11-dihydroxy-4a,8,13,13-tetramethyl-5-oxo-7,11-methano-1H-cyclodeca[3,4]benz[1,2-b]oxet-9-yl ester, [2aR-[2aα,4β,4aβ,6β,9.α(αR*,βS*), 11α,12α,12aα,12bα]]-(9CI) (CAS 151765-61-6)
Benzenepropanoic acid, β-(benzoylamino)-α-hydroxy-,6,12b-bis(acetyloxy)-12-(benzoyloxy)-2a,3,4,4a,5,6,9,10,11,12,12a, 12b-dodecahydro-11-hydroxy-4a,8,13,13-tetramethyl-5-oxo-4-(phosphonooxy)-7,11-methano-1H-cyclodeca[3,4]benz[1,2-b]oxet-9-yl ester, disodium salt,[2aR-2aα,4β,4aβ,6β,9α(αR*,αS*),11α,12α,12aα, 12bα]]-(9CI) (CAS 151695-91-9)
Benzenepropanoic acid, .β-(benzoylamino)-α-(phosphonooxy)-,6,12b-bis(acetyloxy)-12-(benzoyloxy)-2a,3,4,4a,5,6,9,10,11,12,12a,12b-dodecahydro-4,11-dihydroxy-4a,8,13,13-tetramethyl-5-oxo-7,11-methano-1H-cyclodeca[3,4]benz[1,2-b]oxet-9-yl ester, disodium salt, [2aR-[2aα,4β,4aβ,6β,9α(αR*,βS*),11α,12α,12aα,12bα]]-(9CI) (CAS 151695-90-8)

Alfalaxone forms:
5α-Pregnane-11,20-dione, 3β-hydroxy-, dihydrogen phosphate, disodium salt (7CI, 8CI) (CAS 2428-88-8)
(3α, 5α)-3-hydroxypregnane-11,20-dione (CAS 23930-19-0) (Alfaxalone)

The derivative according to the invention when used in any route of administration (oral, transmucosal, intranasal, transdermal, intravenous) may provide increased bioavailability, potential use as a chronic delivery system, increased drug delivery to infected cells, improved membrane transport into virus infected cells and improved lymphatic drug delivery.

The derivative according to the invention in a topical formulation may provide improved dermal & transmucosal penetration, increased systemic bioavailability following dermal delivery, symptomatic relief and reduced viral shedding during treatment with optimized topical formulations.

The derivative according to the invention in an oral formulation may provide improved lymphatic delivery, improved delivery to the brain, lower the loading dose necessary for treatment, lower the incidences of side effects such as constipation, biliary colic, and reduced renal function and decrease inter-patient variability.

The bioavailability of the opioid, steroid hormones, thyroid hormones, anaesthetics or chemotherapeutic agents having a phenolic, primary alcohol, secondary alcohol or tertiary hydroxyl group when provided orally may further benefit from an enteric coating or transfer protein or active domain attachment.

The derivative may be used as a chronic delivery system because of improved dermal penetration and smoother delivery that avoids the peaks and troughs of other delivery routes.

The derivative according to the present invention does not require dissolution in a lipid adjuvant and rapidly reverts to the parent compound upon administration.

When thyroid hormones are administered using the derivative of the present invention, they may have the ability to cross the placenta and appear in breast milk.

### EXAMPLES

The invention will now be further explained and illustrated by reference to the following non-limiting examples.

### Example 1

### Preparation of Phosphatidyl Derivative of Morphine

Morphine hydrochloride 32 g (0.1M) and 37.2 g of sodium valerate (0.3M) were dissolved in 100 ml toluene. 12.6 g (0.05M) of P₄O₁₀ was added and mixed with high shear mixing for one hour slowly raising the temperature to 80° C. 1,2-distearoyl glycerol 30 g was added and the high sheer mixing continued for a further hour at 60° C. 100 ml of a 0.5M sodium hydroxide solution was added and the mixture gently stirred then centrifuged and the process repeated. The toluene phase was recovered and washed with 100 ml of 0.1M hydrochloric acid. The toluene phase was recovered and the toluene and valeric acid removed under vacuum to give 1,2-distearoyl phosphatidyl morphine.

Morphine phosphate was recovered from the aqueous phases.

### Example 2

### Preparation of Complex of Phosphate Derivative of Morphine

12 grams (0.03g/mole) of disodium-N-lauryl beta imino dipropionate were dissolved in 88 grams of distilled water to provide a 12% wt/wt clear solution with pH 12. 11.43 grams (0.03 g/mole) of morphine-3-phosphoric acid ester were slowly added and mixed until uniform. The resulting product was a complex consisting of N-lauryl beta imino dipropionate—morphine (3) phosphate as a 21.03% wt/wt aqueous dispersion. This complex product was formulated via dilution with water preservative buffers together with gelling agents and applied to the skin to elicit transdermal drug delivery.

The complex product may be modified as needed by increasing or decreasing the molar ratio of the disodium-N-lauryl beta imino dipropionate.

### Example 3

### Preparation of Complex of Phosphate Derivative of Paclitaxel

951 g (1 g/mole) of the phosphoric acid ester of Paclitaxel (C₄₇H₅₃NPO₁₈) were complexed with 202 g of lauryl-imino-dipropionate (0.5 g/mole) in 1200 g of deionized water to yield a 49% wt/wt slurry with a pH of 7.5-8.5. Final pH was modified by adding incremental amounts of lauryl-imino-dipropionate.

### Example 4

### Preparation of Complex of Phosphate Derivative of Paclitaxel

174 g (1 g/mole) of arginine was added to 1000 g of deionized water to form a clear solution. 238 g (0.25 g/mole) of the phosphoric ester of paclitaxel was added slowly to form a complex which was 29-30% wt/wt active with a pH of 5-6. The pH was adjusted as desired via adding incremental amounts of arginine or the phosphoric acid ester of paclitaxel.

### Example 5

### Preparation of Complex of Phosphate Derivative of Alfaxalone

860 g (2 g/mole) of the phosphoric acid ester of Alfaxalone (C₂₁H₃₄PO₇) was added to 242.4 g (0.6 g/mole) of disodium lauryl-imino-dipropionate in 2000 ml of deionized water and mixed until homogeneous. The resulting composition is 35-36% solids and had a pH of 4.5-5.5.

### Example 6

### Preparation of Complex of Phosphate Derivative of Alfaxalone

174 g (1 g/mole) of arginine was dissolved in 1000 ml of deionized water and mixed until homogeneous. 430 grams (1 g/mole) of the phosphoric acid ester of Alfaxalone was slowly added with mixing followed by the addition of 500 ml of deionized water to yield a 28-29% active complex with a pH of 6.5-7.5.

The word ‘comprising ’ and forms of the word ‘comprising ’ as used in this description and in the claims does not limit the invention claimed to exclude any variants or additions.

Modifications and improvements to the invention will be readily apparent to those skilled in the art. Such modifications and improvements are intended to be within the scope of this invention.

1. A complex of a pharmaceutical compound selected from the group consisting of opioids, hormones, anaethetics and chemotherapeutic agents comprising the reaction product of:
(a) one or more phosphate derivatives of one or more opioids, steroid hormones, thyroid hormones, anaesthetics or chemotherapeutic agents having a phenolic, primary alcohol, secondary alcohol or tertiary hydroxyl group; and
(b) a complexing agent selected from the group comprising amphoteric surfactants, cationic surfactants, amino acids having nitrogen functional groups and proteins rich in these amino acids.

2. The complex according to claim 1 wherein the phosphate derivative is a phosphatide.

3. An oral formulation comprising a pharmaceutically acceptable carrier and the reaction product of:
(a) one or more phosphate derivatives of one or more opioids, steroid hormones, thyroid hormones, anaesthetics or chemotherapeutic agents having a phenolic, primary alcohol, secondary alcohol or tertiary hydroxyl group; and
(b) a complexing agent selected from the group comprising amino acids having nitrogen functional groups and proteins rich in these amino acids.

4. The oral formulation according to claim 3 wherein the phosphate derivative is a phosphatide.

5. The oral formulation according to claim 3 wherein the complexing agent is selected from the group consisting of glycine, arginine, lysine, histidine, casein and mixtures thereof.

6. The oral formulation according to claim 3 further comprising an effective amount of the reaction product of:
(a) one or more phosphate derivatives of tocopherol; and
(b) a complexing agent selected from the group comprising amino acids having nitrogen functional groups and proteins rich in these amino acids.

7. The oral formulation according to claim 3 further comprising an enteric coating.

8. The oral formulation according to claim 3 further comprising a transfer protein or active domain attachment

9. A phosphatidyl derivative of a pharmaceutical compound selected from the group consisting of opioids, steroid hormones, thyroid hormones, anaesthetics or chemotherapeutic agents having a phenolic, primary alcohol, secondary alcohol or tertiary hydroxyl group.

10. The phosphatidyl derivative of a pharmaceutical compound according to claim 9 wherein the phosphatidyl group is complexed with a complexing agent selected from the group comprising amphoteric surfactants, cationic surfactants, amino acids having nitrogen functional groups and proteins rich in these amino acids.

11. A method for preparation of a phosphate derivative of a pharmaceutical compound selected from the group consisting of opioids, steroid hormones, thyroid hormones, anaesthetics or chemotherapeutic agents having a phenolic, primary alcohol, secondary alcohol or tertiary hydroxyl group comprising the step of reacting the pharmaceutical compound with P₄O₁₀ in the presence of a sodium salt of a fatty acid.

12. The method according to claim 11 further comprising the step of reacting the product from the P₄O₁₀ reaction with a di or mono acyl glyceride to form a phosphatide.

13. The method according to claim 11 wherein the pharmaceutical compound is selected from the group consisting of morphine (CAS 57-27-2), hydromorphone, oxymorphone, levorphanol, codeine, oxycodone, nalbuphine, buprenorphine, butorphanol, pentazocine, nalorphine (CAS 62-67-9), naloxone, naltrexone, levallorphan, levothyroxine (CAS 51-48-9), paclitaxel (CAS 33069-62-4), alfaxalone (CAS 23930-19-0), estradiol (CAS 50-28-2), estrone (CAS 53-16-7), estriol (CAS 50-27-1), ethinyl estradiol, progestins, methyltestosterone, testosterone (CAS 58-22-0), nandrolone (CAS 434-22-0) and danazol.

14. A phosphate derivative of a pharmaceutical compound selected from the group consisting of opioids, steroid hormones, thyroid hormones, anaesthetics or chemotherapeutic agents having a phenolic, primary alcohol, secondary alcohol or tertiary hydroxyl group.

15. The phosphate derivative according to claim 14 wherein the phosphate derivative is a phosphatidyl derivative.

16. Use of a phosphate derivative of a pharmaceutical compound selected from the group consisting of opioids, steroid hormones, thyroid hormones, anaesthetics or chemotherapeutic agents having a phenolic, primary alcohol, secondary alcohol or tertiary hydroxyl group to make a medicament for use in treating humans.

17. Use of a phosphate derivative of a pharmaceutical compound selected from the group consisting of opioids, steroid hormones, thyroid hormones, anaesthetics or chemotherapeutic agents having a phenolic, primary alcohol, secondary alcohol or tertiary hydroxyl group to make a medicament for use in treating animals.
